(19) United States
(12) Patent Application Publication
(10) Pub. No.: US 2023/0414175 A1
(43) Pub. Date: Dec. 28, 2023

(54) A PATCH FOR APPLICATION TO HUMAN OR ANIMAL ORGAN AND A PROCESS FOR MANUFACTURING THEREOF

(71) Applicants: CAMLIN ITALY S.r.l., Parma (IT); CONSIGLIO NAZIONALE DELLE RICERCHE, Roma (IT)

(72) Inventors: Giuseppe TARABELLA, Collecchio (Parma) (IT); Luca ASCARI, Parma (IT); Stefano LAI, Parma (IT); Konstantin KOSHMAK, Trieste (IT); Davide VURRO, Parma (IT); Pasquale D’ANGELO, Langhirano (Parma) (IT); Salvatore IANNOTTA, Trento (IT)

(21) Appl. No.: 18/253,368

(22) PCT Filed: Nov. 15, 2021
(86) PCT No.: PCT/IB2021/060560

§ 371 Date: May 17, 2023

(30) Foreign Application Priority Data
Nov. 18, 2020 (IT) 102020000027690

(51) Int. Cl. A61B 5/00; A61B 5/268; A61B 5/257; C09D 11/04; C09D 11/52; C07K 14/435

(52) U.S. Cl. CPC A61B 5/6833; A61B 5/268; A61B 5/257; C09D 11/04; C09D 11/52; C07K 14/43586; A61B 2562/125; A61B 2560/0412

(57) ABSTRACT

A process (100) for manufacturing a patch (10) to be applied on a human or animal organ, comprising the steps of:
preparing a bio-polymeric ink (200);
providing a substrate at least partially made by said bio-polymeric ink (300);
printing a circuit on said substrate (400).

### TECHNICAL FIELD

The present invention relates to a patch for application to human or animal organ and a process for manufacturing thereof.

The proposed invention finds application in bioelectrical and chemical sensing. For example, the invention may be used in wearable sensor devices which enable continuous monitoring of biological or chemical information from the human body, either for medical applications, or in healthcare or sports.

In these sectors, soft and stretchable electronic devices are preferred to traditional electronic sensors since they better match the mechanical properties of human organs like skin and brain, assuring a higher level of comfort and reliability even during motion of the patient.

### BACKGROUND ART

There are already known on the market soft electronic devices that are based on biomaterial such as silk, cellulose, chitin, and lignin.

Most of the substrates currently available require glues or gels for enhancing adhesion on the skin but tend to dry out very quickly, that causes a detachment of the electrodes and deterioration of the interfacial impedance properties.

Silk fibroin is an ideal platform for wearable electronic devices and implantable applications thanks to the natural biocompatibility and biodegradability.

It is already known a method for plasticizing the original hard silk fibres into soft and stretchable films by introducing CaCl₂) into silk and controlling the relative humidity of the moisture.

Even if this method allows to obtain a fibroin substrate having higher adhesion properties on skin without the use of gel or glues, it still poses issues relating to mechanical stability for hosting 3D printed circuits and electrodes made of metallic or organic material.

In addition, printing metal connections on a fibroin substrate is critical. Lithography is the most popular technique used to print metallic lines, whereas spin-coating, ink-jet printing and tattoo transfer are the mainly used methods for organic material. Nevertheless, most of the 3D printing organic materials need to be annealed at a high temperature that is not compatible with the physical and chemical properties of the fibroin, causing its degradation.

### DISCLOSURE OF THE INVENTION

In this context, the technical task at the basis of the present invention is to propose a patch for application to human or animal organ and a process for manufacturing thereof, which overcome the above-mentioned drawbacks of the prior art.

In particular, the object of the present invention is to propose a patch for application to human or animal organ having both good adhesion properties on the skin and offering a stable support for 3D printed circuits.

Another object of the present invention is to propose a patch for application to human or animal organ, that is biocompatible and that reduces or eliminates clinical wastes.

Another object of the present invention is to propose a patch that is versatile, that means it reliably supports different types of devices for sensing chemical or biological properties of human or animal organs.

Another object of the present invention is to propose a process for manufacturing a patch for application to human or animal organ, that overcomes the compatibility issues between different materials that are still present in prior art solutions (e.g. printing metal lines on fibroin).

The stated technical task and specified objects are substantially achieved by a process for manufacturing a patch for to be applied on a human or animal organ, comprising the steps of:
preparing a bio-polymeric ink;
providing a substrate at least partially made by the bio-polymeric ink;
printing a circuit on the substrate.

According to one aspect of the invention, the step of preparing the bio-polymeric ink comprises extracting fibroin from Bombyx mori cocoons.

In accordance with one embodiment, the step of preparing the bio-polymeric ink further comprises boiling the cocoons in a solution of distilled or deionized water and sodium carbonate so as to obtain a degummed fibroin.

In accordance with one embodiment, the step of preparing the bio-polymeric ink further comprises dissolving the degummed fibroin in a solution of formic acid and Calcium chloride.

In accordance with one aspect of the invention, the solution of formic acid and Calcium chloride comprises also one or more of the following: Sodium Chloride, Potassium Chloride, Magnesium Chloride.

In accordance with one aspect of the invention, the solution of formic acid and Calcium chloride comprises also a Carboxylic acid.

According to one embodiment of the invention, the step of providing the substrate is carried out by aerosol jet printing of the bio-polymeric ink.

According to another embodiment, the step of providing the substrate is carried out by inkjet printing or screen printing or roll-to-roll of the bio-polymeric ink.

According to another embodiment, the step of providing the substrate is carried out by drop-casting or spin coating of the bio-polymeric ink.

According to one aspect of the invention, the bio-polymeric ink is prepared by a solution of one of the following: a Poly-Lactic-co-Glycolic Acid, Poly-Capro-Lactone, Chitosan, Dextrin, Poly Lactic Acid, Polyglicolic Acid, Ethyl Cellulose, Hydroxypropil MethylCellulose, Hydroxyethyl methyl cellulose.

According to one aspect of the invention, the step of printing a circuit on the substrate comprises:
printing a metal circuit on a Silicon wafer;
transferring the printed metal circuit from the Silicon wafer on the substrate.

According to one embodiment, the step of printing of the metal circuit on a Silicon wafer consists in aerosol jet printing.

According to one embodiment, the step of printing a circuit on the substrate is carried out by aerosol jet printing of an organic semiconductor on the substrate.

For example, the organic semiconductor is a conducting polymer.

In accordance with one aspect of the invention, the process further comprises a step of depositing a protective layer on the circuit.

In particular, the step of depositing the protective layer is carried out by aerosol jet printing.

The stated technical task and specified objects are substantially achieved by a patch for application to human or animal organ, comprising:
a substrate having a substantially flat shape;
a contact zone with the organ, the contact zone being obtained on a first surface of the substrate and comprising a bio-polymeric ink;
a circuit hosted on the substrate, that is opposite to said first surface.

In accordance with one embodiment, the circuit is hosted on a second surface of the substrate, that is opposite to the first surface.

In accordance with one embodiment, the patch further comprises a protective layer deposited on the circuit.

For example, the bio-polymeric ink is based on fibroin.

Other alternative of the bio-polymeric ink is that it is based on one of the following: a Poly-Lactic-co-Glycolic Acid, PolyCaproLactone, Chitosan, Dextrin, Poly Lactic Acid, Polyglicolic Acid, Ethyl Cellulose, Hydroxypropil MethylCellulose, Hydroxyethyl methyl cellulose.

In accordance with one embodiment, the circuit is made of a metal and/or organic semiconductor.

In accordance with one aspect of the invention, the circuit is configured to detect a bioelectrical signal from a human or animal body.

In accordance with one aspect of the invention, the circuit is configured to sense a biochemical property of a human or animal body.

### BRIEF DESCRIPTION OF DRAWINGS

Further characteristics and advantages of the present invention will more fully emerge from the non-limiting description of a preferred but not exclusive embodiment of a patch for application to human or animal organ and a process for manufacturing thereof, as depicted in the attached figures:

FIG. 1 illustrates a flow-chart of a process for manufacturing a patch for application to human or animal organ, according to the present invention;

FIGS. 2 and 3 illustrate flow-charts of the process of FIG. 1, according to different embodiments;

FIG. 4 schematically illustrates a patch for application to human or animal organ, according to the present invention, respectively in a side view (a), a top view (b) and a bottom view (c).

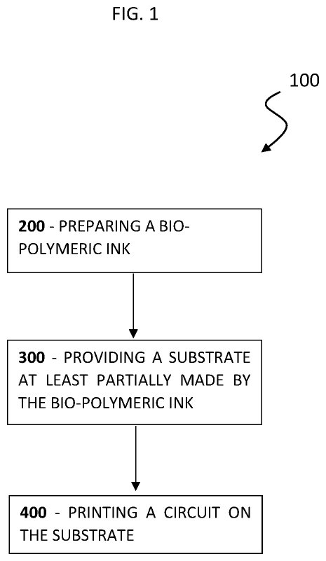

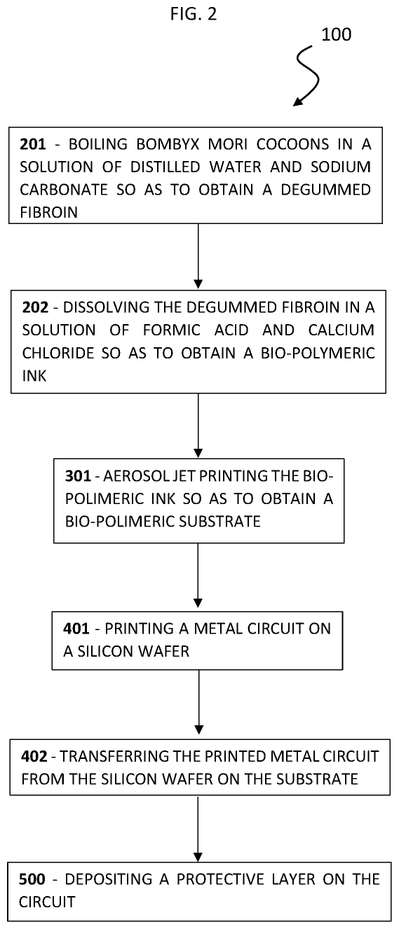

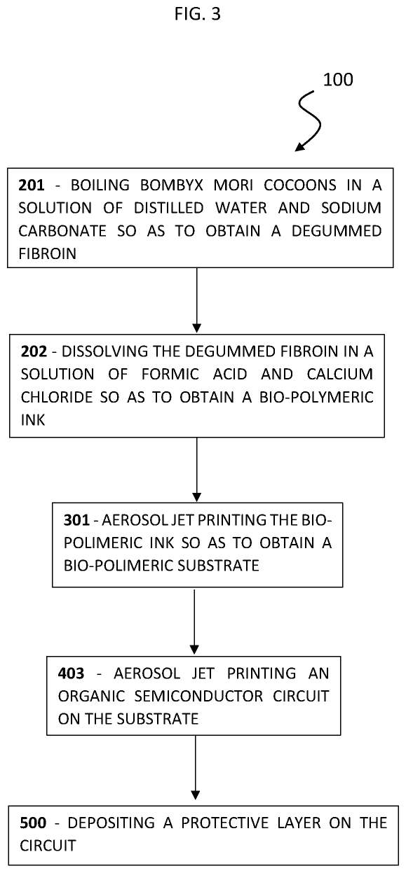

### DETAILED DESCRIPTION OF PREFERRED EMBODIMENTS OF THE INVENTION

The process 100 starts with a step of preparing a bio-polymeric ink (block 200).

According to one aspect of the invention, the bio-polymeric ink is fibroin-based. Thus, the preparation of the bio-polymeric ink comprises extracting fibroin from Bombyx mori cocoons.

In particular, the cocoons are first cut in pieces, for example using a titanium scissor, and then boiled in a solution of distilled or deionized water and sodium carbonate (block 201) in order to remove the glue-like cladding made of sericin, that would cause irritation on the skin.

The result of the boiling step is a degummed fibroin that is then dissolved in a solution of formic acid and Calcium chloride (block 202).

According to one aspect of the invention, salts are added to the solution of formic acid and Calcium chloride for tuning the adhesion properties and viscosity of the final ink.

For example, hydroscopic salts may be added in the concentration from 1 to 40% w/v of the whole solution.

For example, one or more of the following can be added to the solution of formic acid and Calcium chloride: Sodium Chloride (NaCl), Potassium Chloride (KCl), Magnesium Chloride (MgCl₂) or other hygroscopic salts.

According to an embodiment of the invention, Carboxylic acids such as ethanoic acid, propanoic, butanoic acid, pentanoic acid, etc., may be used for the fibroin dissolution from its degummed phase. The concentration of Carboxylic acids spans preferably from 1 to 30% w/v.

In order to obtain a fast and homogeneous dissolution, the fibroin solution is stirred.

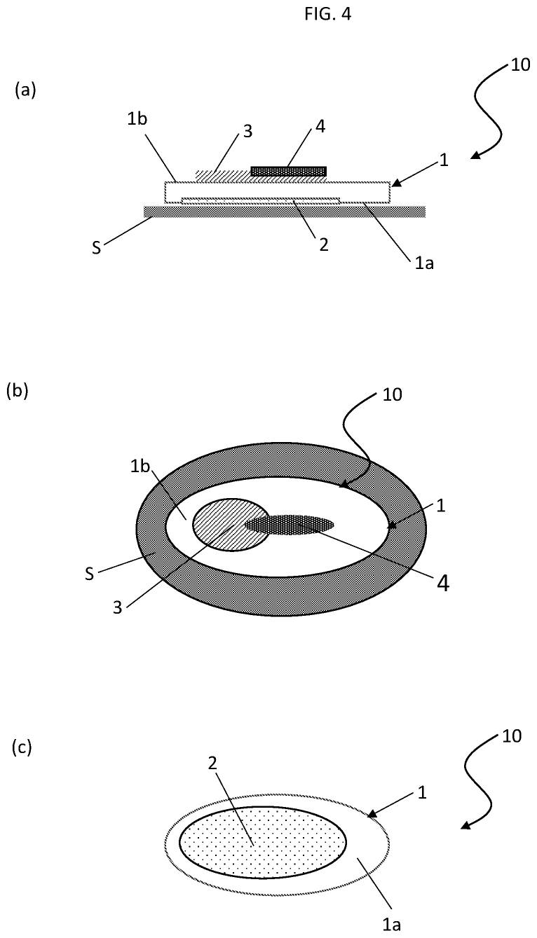

The bio-polymeric ink resulting from the steps above is then used to create a substrate (block 300), indicated with number 1 in FIG. 4.

According to an embodiment, the substrate is obtained by aerosol jet printing of the bio-polymeric ink (block 301).

In particular, by aerosol jet printing the fibroin ink, the substrate is obtained in the form of a fibroin film having a thickness equal or lower than 50 um.

According to another embodiment, the substrate is obtained by inkjet printing or screen printing or roll-to-roll of the bio-polymeric ink.

According to another embodiment, the substrate is obtained by deposition of the bio-polymeric ink, for example by drop-casting (e.g. in a Petri dish) or by spin coating.

Preferably, during the formation of the substrate both temperature and humidity of the environment are kept in controlled ranges. For example, the temperature is comprised between 20° C. and 30° C. The humidity is kept in the range of RH 40-60%.

Preferably, the substrate 1 has a substantially flat shape having a first surface 1a and a second surface 1b opposite to the first surface 1a.

According to one example, the first surface 1a and the second surface 1b are parallel.

At least one area of the first surface 1a comprises the bio-polymeric ink. This area constitutes a contact zone with the skin or with another organ of a human or animal body. The contact zone is indicated with number 2 in FIG. 4. In FIG. 4 it is shown the first surface 1a adhering to a portion of skin, indicated with S.

The extension of the contact zone 2 may vary depending on the specific application.

According to an embodiment of the invention, the contact zone 2 has an extension that is lower than the extension of the first surface 1a.

According to another embodiment, the contact zone 2 coincides with the whole first surface 1a.

According to one embodiment, the substrate 1 is made of a single fibroin layer with different ions gradient concentration in the first surface 1a and in the second surface 1b. These surfaces are in fact treated with specific ionic liquid or other substance creating transfer of ions locally at the interface of area of interest.

According to one aspect, the fibroin can be made with a gradient of concentration obtained by tuning the concentration of hygroscopic ions used during the extraction of fibroin, such as Ca2+, K+, Na+, Mg2+.

For example, concentration of hygroscopic ions is comprised in the range 1-40 wt %.

Different concentration of hygroscopic ions in fibroin films changes important properties of the material, such as mechanical responses (sturdiness and adhesivity) and electrical conductivity.

Another alternative of obtaining ions gradient distribution is by modifying locally the ions concentration of the single layer, thus in the first surface 1a and in the second surface 1b.

According to one example, this may be done by printing locally—over one of the surfaces of the single layer—a fibroin ink at a different ion concentration.

According to another example, this may be done by printing locally along the circular edge over one of the surfaces of the single layer, a fibroin ink at a different ion concentration.

Alternative to the fibroin, the bio-polymeric ink may be based on a solution of PLGA (acronym for Poly-Lactic-co-Glycolic Acid) or PLC (acronym for Poly-Capro-Lactone) or Chitosan or Dextrin or PLA (acronym for Poly Lactic Acid) or PGA (acronym of Polyglicolic Acid) or Ethyl Cellulose, or Hydroxypropil MethylCellulose or Hydroxyethyl methyl cellulose.

The following part of description is relative to the embodiment of substrate 1 made of a single layer, with the first surface 1 and the second surface 1b.

After the substrate 1 is obtained, a circuit 3 is printed on one of the surfaces 1a, 1b of the substrate 1 (block 400).

According to an embodiment of the invention, the circuit 3 is printed on the second surface 1b of the substrate 1.

According to another embodiment of the invention, the circuit 3 is printed on the first surface 1a of the substrate 1.

According to another embodiment of the invention, on both the surfaces 1a, 1b of the substrate 1 there is printed a corresponding circuit.

The circuit can be made of metal or organic material or both.

According to one aspect of the invention, printing of a metal circuit comprises:
printing the metal circuit on a Silicon wafer (block 401);
transferring the printed metal circuit from the Silicon wafer on the substrate 1 (block 402).

In particular, printing of the metal circuit on the Silicon wafer is done by aerosol jet printing of metals, sintered at a temperature in the range of 100° C.-250° C., for 20-60 minutes.

The transfer printing of the metal circuit on the fibroin film (or, generically, on the bio-polymeric area of the substrate 1) is done at a temperature lower than 40° C., for example at room temperature.

According to one aspect of the invention, the circuit 3 comprises an organic semiconductor that is printed on the substrate 1 by aerosol jet printing (block 403), in particular at a temperature lower than 25° C.

Usually, aerosol jet printing at temperature lower than 25° C. is referred to as “cold” aerosol jet printing.

In particular, the organic semiconductor is a conducting polymer. For example, the organic material can be p-type polythiopene, such as PEDOT (poly(3,4-ethylenedioxythiophene), P3HT, PTHS, PANI or PPy. Also, the organic material can be a n-type semiconductor based on imide derivates copolymerized with electron donor (thiopene derivates) units or electron-deficient units (bithiazole, benzothiazole, azine, etc.).

The PEDOT may also be doped with anions such as PSS (PEDOT:PSS), Tosylate (PEDOT:PSS), metallic cations (such as Na+), poly(ethylene glycol) (PEG) and solution processable n-type conductors, such as polyimides.

Also, secondary doping molecules may be used for tuning the conductivity. The organic semiconductor is printed in one terminal passive electrode and three-terminal active (e.g. transistor-like) electrical configuration.

In this context, the term circuit refers to a path or paths that allows current to flow from one point to another. A circuit may also comprise one or more devices, either made by inorganic or organic materials.

According to one aspect of the invention, the circuit 3 of the invention comprises one or more sensors, i.e. chemical or biochemical sensors, that are configured to perform specific measurements, depending on the application of the patch.

According to another embodiment (not illustrated), the substrate 1 comprises a plurality of overlapped/stacked layers starting from the bio-polymeric ink.

The composition of the layers may be singly tuned so as to confer higher adhesive properties or higher structural properties or both to each layer.

In fact, ions gradient distribution may be obtained by using overlapped or stacked layers with different ions concentration levels.

According to one embodiment, the substrate 1 comprises two layers of bio-polymeric ink (based on fibroin), each layer having a different ions concentration.

In particular, the substrate 1 comprises:
a first layer with a higher ions concentration (for example at 30%);
a second layer with an ions concentration that is lower than the first layer (for example at 20%).

The first layer shows higher adhesive properties on the skin than the second layer. Thus, the first layer is usually used as adhesive layer.

The second layer, being more rigid than the first layer, offers a primary structural function.

According to a variant, the substrate 1 comprises also a third layer, which is referred herein as “electronics layer”. This electronics layer is added on the top of the second layer.

In particular, the first layer is obtained by aerosol jet printing the fibroin ink. The second layer is also obtained by aerosol jet printing and deposited on the first layer.

According to an aspect, at least one hole may be created within the second layer.

The hole may be created by removing material from the second layer or by depositing the second layer in a controlled way so that a hole is obtained therein. Another way to obtain the hole is by using a specific mould.

The hole may be filled by another material, for example by deposition. In this context, the filled hole is referred to as a “channel”.

The creation of the channel results in improving impedance matching and conductivity.

For example, the channel is made of an organic polymer.

In another example, the channel is made of the same fibroin of the first layer, thus having a higher ions concentration.

In particular, the electronics layer is composed of conductive interconnects or other electronics components, for example printed over a strip of Parylene. In practice, the electronics layer hosts the circuit 3.

Thus, the interconnects are in contact with the exposed surface area of the channel material. It is this matching area that justifies the fabrication of the hole in the second layer structure.

In fact, the reason of adding the channel is to link the first layer with the top electronics layer, because of the high electrical resistivity of the second layer surrounding the channel and causing a high voltage-drop if not by-passed.

In another variant, the electronics layer is composed by a conductive connector (e.g. a metallic button), whose size is matching the size of the hole in the second layer. The connector can allow for matching the current commercial standard equipment.

According to a variant, there could be obtained a plurality of holes in the second layer. These holes are filled with another material to create a plurality of channels.

As previously said, the substrate 1 may also comprise more than two fibroin layers, on the top of the last one being added an electronics layer similar to the one already described.

A non-limiting list of applications of the patch is listed below.
Detection of bioelectrical signals from human body, such as Electroencephalogram (EEG), Electrocardiogram (ECG), Electromyogram (EMG), Mechanomyogram (MMG), Electrooculography (EOG), Galvanic skin response (GSR), Magnetoencephalogram (MEG).
Detection of chemicals (such as dopamine, glucose, sodium) in physiological fluids such as saliva, sweat, blood, etc.
Detection of biological cells (e.g. bacteria) in physiological fluids such as saliva, sweat, blood, etc.
Hosting biochemical sensor endowed with highly selective biochemical properties. In one case, selectivity could be achieved through the antigen-antibody recognition system, where the antigen is attached on the surface of a gate terminal under a three-terminal transistor architecture. In another case, the antigen could be loaded on the surface of a single-terminal electrode. In some cases, the selective elements could be DNA segments or synthetic DNA segments (aptamers), built toward a specific protein or molecular recognition, such as infectious agent (e.g. virus) in physiological fluids (such as sweat, blood).
Hosting a sensor with a fork-like shape layout designed for bioelectrical signals. According to one design option, the bioelectrical sensor has a dedicated layout, specifically planned and designed for amplification of bioelectrical signals, with low amplitude (e.g. below 1 μV). The design may also be a fork-like pattern for the simultaneous detection of bioelectrical signals from multiple closed point on the body (e.g. EEG on the scalp). According to another design option, the sensor can be designed with a fractal structure for the amplification of a single point detection.
Hosting a biopotential sensor with a fractal structure designed for recording and amplifying bioelectrical and chemical signals. In this context, the expression “fractal structure” means that the shape of the transducer element is replicated also in the configuration of the multi-sites (array) transducer while keeping a subset of contacts short circuited.
Hosting an organic transistor, an organic field-effect transistor, or an organic electrochemical transistor, with amplifying properties.

A protective layer 4, for example a synthetic dielectric material, is preferably deposited on the circuit 3 (block 500), either metal or organic. For the deposition of the protective layer 4, the preferred technique is aerosol jet printing at a temperature lower than 25° C.

The characteristics and the advantages of a patch for application to human or animal organ and of a process for manufacturing thereof, according to the present invention, are clear, as are the advantages.

The overall process allows the manufacturing of flexible natural substrates hosting 3D printed active and/or passive organic semiconductors and/or electrical circuits for different applications.

The substrate of the patch is partially or wholly made by the printable bio-polymeric ink, and can work as:
a “structural layer” that hosts printed transducers, electrodes, interconnects;
an “adhesive layer” on the skin or other organs, allowing a durable adhesion property that can be simply regenerated by using water;
an “impedance matching layer”, making the impedance between the circuit and the skin stable over time.

As already pointed out, the main interface with the skin is made of fibroin or other bio-polymeric material, that is a full biocompatible material.

The substrate may be completely dissolved under water flow, so it is green disposable. It is sufficient to have even a small area of contact with the skin for obtaining adhesion.

The bio-polymeric ink has been optimized for being deposited or printed. through aerosol jet. In particular, the recipe of extraction of the fibroin has been optimized to produce a flexible substrates and to obtain an ideal adhesion of the patch on the skin, i.e. an adhesion that is both stable enough during daily life activity and not too strong to cause pain under detachment of the patch.

By varying the salts concentration, the final mechanical properties of the ink may be tuned in terms of stretchability, flexibility and self-adhesion on human skin. In addition, the aerosol jet printing of the metal circuit on the Silicon wafer and the subsequent transferring on the fibroin substrate overcomes the compatibility issue of direct printing metal layout on a fibroin substrate.

Indeed, the fibroin substrate dries and damages at elevated temperatures (higher than 40° C.) in 1-10 minutes depending on the temperature, but also at temperatures lower than 40° C. for longer time, thus it is not compatible with aerosol printing of metal inks made of metallic nanoparticle dispersed in solvents and co-solvents. The latter in fact require high temperature for the evaporation, as well as for promoting sintering. It must be pointed out that metals like Ag, Au, Cu shows very-low adhesion on the surface of the Silicon wafer. This low adhesion, that is indeed a drawback in a standard lithographic process, facilitates here the mechanical transfer of metal circuits on the fibroin substrate.

The specific substrate composed by two fibroin layers is particularly advantageous since it allows to enhance the structural function of one layer and the adhesive properties of the other layer.

1. A process for manufacturing a patch to be applied on a human or animal organ, comprising the steps of:
preparing a bio-polymeric ink;
providing a substrate at least partially made by said bio-polymeric ink (300);
printing a circuit on said substrate.

2. The process of claim 1, wherein the step of preparing the bio-polymeric ink comprises extracting fibroin from Bombyx mori cocoons.

3. The process of claim 2, wherein the step of preparing the bio-polymeric ink further comprises boiling said cocoons in a solution of distilled or deionized water and sodium carbonate so as to obtain a degummed fibroin.

4. The process of claim 3, wherein the step of preparing the bio-polymeric ink further comprises dissolving the degummed fibroin in a solution of formic acid and Calcium chloride.

5. The process of claim 4, wherein the solution of formic acid and Calcium chloride comprises also one or more of the following: Sodium Chloride, Potassium Chloride, Magnesium Chloride.

6. The process of claim 4, wherein the solution of formic acid and Calcium chloride comprises also a Carboxylic acid.

7. The process of claim 1, wherein the step of providing the substrate is carried out by aerosol jet printing of the bio-polymeric ink.

8. The process of claim 1, wherein the step of providing the substrate is carried out by inkjet printing or screen printing or roll-to-roll of the bio-polymeric ink.

9. The process of claim 1, wherein the step of providing the substrate is carried out by drop-casting or spin coating of the bio-polymeric ink.

10. The process of claim 1, wherein the bio-polymeric ink is prepared by a solution of one of the following: a Poly-Lactic-co-Glycolic Acid, Poly-Capro-Lactone, Chitosan, Dextrin, Poly Lactic Acid, Polyglicolic Acid, Ethyl Cellulose, Hydroxypropil MethylCellulose, Hydroxyethyl methyl cellulose.

11. The process of claim 1, wherein the step of printing a circuit on the substrate comprises:
printing a metal circuit on a Silicon wafer;
transferring the printed metal circuit from the Silicon wafer on the substrate.

12. The process of claim 11, wherein printing of the metal circuit on a Silicon wafer consists in aerosol jet printing.

13. The process of claim 1, wherein the step of printing a circuit on the substrate is carried out by aerosol jet printing of an organic semiconductor on said substrate.

14. The process of claim 13, wherein the organic semiconductor is a conducting polymer.

15. The process of claim 1, further comprising a step of depositing a protective layer on the circuit.

16. The process of claim 15, wherein the step of depositing the protective layer is carried out by aerosol jet printing.

17. A patch for application to human or animal organ, comprising:
a substrate having a substantially flat shape;
a contact zone with the organ, said contact zone being obtained on a first surface of the substrate and comprising a bio-polymeric ink;
a circuit hosted on the substrate, that is opposite to said first surface.

18. The patch of claim 17, wherein the circuit is hosted on a second surface of the substrate, that is opposite to said first surface.

19. The patch of claim 17, further comprising a protective layer deposited on said circuit.

20. The patch of claim 17, wherein the bio-polymeric ink is based on fibroin.

21. The patch of claim 17, wherein the bio-polymeric ink is based on one of the following: a Poly-Lactic-co-Glycolic Acid, PolyCaproLactone, Chitosan, Dextrin, Poly Lactic Acid, Polyglicolic Acid, Ethyl Cellulose, Hydroxypropil MethylCellulose, Hydroxyethyl methyl cellulose.

22. The patch of claim 17, wherein the circuit is made of a metal and/or organic semiconductor.

23. The patch of claim 17, wherein the circuit is configured to detect a bioelectrical signal from a human or animal body.

24. The patch of claim 17, wherein the circuit is configured to sense a biochemical property of a human or animal body.
